(19) United States
(12) Patent Application Publication
(10) Pub. No.: US 2001/0012513 A1
(43) Pub. Date: Aug. 9, 2001

(54) Embryonic or stem-like cell lines produced by cross species nuclear transplantation

(75) Inventors: James Robl, Belchertown, MA (US); Jose Cibelli, Amherst, MA (US); Steven L. Stice, Belchertown, MA (US)

Correspondence Address: Pillsbury Winthrop LLP, Intellectual Property Group, East Tower, Ninth Floor, 1100 New York Avenue, N.W., Washington, DC, 20005-3918, US

(73) Assignee: University of Massachusetts

(21) Appl. No.: 09/809,018

(22) Filed: Mar. 16, 2001

Related U.S. Application Data
(63) Continuation of application No. 09/032,945, filed on Mar. 2, 1998, now abandoned.
Application No. 09/032,945 is a continuation-in-part of application No. 08/699,040, filed on Aug. 19, 1996.

(51) Int. Cl. A61K 48/00; C12N 5/08

(52) U.S. Cl. 424/93.21; 435/366

(57) ABSTRACT

An improved method of nuclear transfer involving the transplantation of donor cell nuclei into enucleated oocytes of a species different from the donor cell is provided. The resultant nuclear transfer units are useful for the production of isogenic embryonic stem cells, in particular human isogenic embryonic or stem cells. These embryonic or stem-like cells are useful for producing desired differentiated cells and for introduction, removal or modification, of desired genes, e.g., at specific sites of the genome of such cells by homologous recombination. These cells, which may contain a heterologous gene, are especially useful in cell transplantation therapies and for in vitro study of cell differentiation.

### CROSS-REFERENCE TO RELATED APPLICATIONS

This application is a continuation of U.S. Ser. No. 09/032,945, filed Mar. 2, 1998, which is a continuation-in-part of U.S. Ser. No. 08/699,040, filed Aug. 19, 1996. These applications are incorporated by reference in their entirety herein.

### FIELD OF THE INVENTION

The present invention relates to the production of embryonic or stem-like cells by transplantation of cell nuclei derived from animal or human cells into enucleated animal oocytes of a species different from the donor nuclei. The present invention more specifically relates to the production of human embryonic or stem-like cells by transplantation of the nucleus of a human cell into an enucleated animal oocyte, preferably an ungulate oocyte and most preferably a bovine enucleated oocyte.

The present invention further relates to the use of the resultant embryonic or stem-like cells, preferably human embryonic or stem-like cells for therapy, for diagnostic applications, for the production of differentiated cells which may also be used for therapy or diagnosis, and for the production of transgenic embryonic or transgenic differentiated cells, cell lines, tissues and organs. Also, the embryonic or stem-like cells obtained according to the present invention may themselves be used as nuclear donors in nuclear transplantation or nuclear transfer methods.

### BACKGROUND OF THE INVENTION

Methods for deriving embryonic stem (ES) cell lines in vitro from early preimplantation mouse embryos are well known. (See, e.g., Evans et al., Nature, 29:154-156 (1981); Martin, Proc. Natl. Acad. Sci., USA, 78:7634-7638 (1981)). ES cells can be passaged in an undifferentiated state, provided that a feeder layer of fibroblast cells (Evans et al., Id.) or a differentiation inhibiting source (Smith et al., Dev. Biol., 121:1-9 (1987)) is present.

ES cells have been previously reported to possess numerous applications. For example, it has been reported that ES cells can be used as an in vitro model for differentiation, especially for the study of genes which are involved in the regulation of early development. Mouse ES cells can give rise to germline chimeras when introduced into preimplantation mouse embryos, thus demonstrating their pluripotency (Bradley et al., Nature, 309:255-256 (1984)).

In view of their ability to transfer their genome to the next generation, ES cells have potential utility for germline manipulation of livestock animals by using ES cells with or without a desired genetic modification. Moreover, in the case of livestock animals, e.g., ungulates, nuclei from like preimplantation livestock embryos support the development of enucleated oocytes to term (Smith et al., Biol. Reprod., 40:1027-1035 (1989); and Keefer et al., Biol. Reprod., 50:935-939 (1994)). This is in contrast to nuclei from mouse embryos which beyond the eight-cell stage after transfer reportedly do not support the development of enucleated oocytes (Cheong et al, Biol. Reprod., 48:958 (1993)). Therefore, ES cells from livestock animals are highly desirable because they may provide a potential source of totipotent donor nuclei, genetically manipulated or otherwise, for nuclear transfer procedures.

Some research groups have reported the isolation of purportedly pluripotent embryonic cell lines. For example, Notarianni et al., J. Reprod. Fert. Suppl., 43:255-260 (1991), report the establishment of purportedly stable, pluripotent cell lines from pig and sheep blastocysts which exhibit some morphological and growth characteristics similar to that of cells in primary cultures of inner cell masses isolated immunosurgically from sheep blastocysts. (Id.) Also, Notarianni et al., J. Reprod. Fert. Suppl., 41:51-56 (1990) discloses maintenance and differentiation in culture of putative pluripotential embryonic cell lines from pig blastocysts. Further, Gerfen et al., Anim. Biotech, 6(1):1-14 (1995) disclose the isolation of embryonic cell lines from porcine blastocysts. These cells are stably maintained in mouse embryonic fibroblast feeder layers without the use of conditioned medium. These cells reportedly differentiate into several different cell types during culture (Gerfen et al., Id.).

Further, Saito et al., Roux's Arch. Dev. Biol., 201:134-141 (1992) report bovine embryonic stem cell-like cell lines cultured which survived passages for three, but were lost after the fourth passage. Still further, Handyside et al., Roux's Arch. Dev. Biol., 196:185-190 (1987) disclose culturing of immunosurgically isolated inner cell masses of sheep embryos under conditions which allow for the isolation of mouse ES cell lines derived from mouse ICMs. Handyside et al. (1987) (Id.), report that under such conditions, the sheep ICMs attach, spread, and develop areas of both ES cell-like and endoderm-like cells, but that after prolonged culture only endoderm-like cells are evident. (Id.)

Recently, Chemy et al., Theriogenology, 41:175 (1994) reported purportedly pluripotent bovine primordial germ cell-derived cell lines maintained in long-term culture. These cells, after approximately seven days in culture, produced ES-like colonies which stain positive for alkaline phosphatase (AP), exhibited the ability to form embryoid bodies, and spontaneously differentiated into at least two different cell types. These cells also reportedly expressed mRNA for the transcription factors OCT4, OCT6 and HES1, a pattern of homeobox genes which is believed to be expressed by ES cells exclusively.

Also recently, Campbell et al., Nature, 380:64-68 (1996) reported the production of live lambs following nuclear transfer of cultured embryonic disc (ED) cells from day nine ovine embryos cultured under conditions which promote the isolation of ES cell lines in the mouse. The authors concluded based on their results that ED cells from day nine ovine embryos are totipotent by nuclear transfer and that totipotency is maintained in culture.

Van Stekelenburg-Hamers et al., Mol. Reprod. Dev., 40:444-454 (1995), reported the isolation and characterization of purportedly permanent cell lines from inner cell mass cells of bovine blastocysts. The authors isolated and cultured ICMs from 8 or 9 day bovine blastocysts under different conditions to determine which feeder cells and culture media are most efficient in supporting the attachment and outgrowth of bovine ICM cells. They concluded based on their results that the attachment and outgrowth of cultured ICM cells is enhanced by the use of STO (mouse fibroblast) feeder cells (instead of bovine uterus epithelial cells) and by the use of charcoal-stripped serum (rather than normal serum) to supplement the culture medium. Van Stekelenburg et al reported, however, that their cell lines resembled epithelial cells more than pluripotent ICM cells. (Id.)

Still further, Smith et al., WO 94/24274, published Oct. 27, 1994, Evans et al, WO 90/03432, published Apr. 5, 1990, and Wheeler et al, WO 94/26889, published Nov. 24, 1994, report the isolation, selection and propagation of animal stem cells which purportedly may be used to obtain transgenic animals. Also, Evans et al., WO 90/03432, published on Apr. 5, 1990, reported the derivation of purportedly pluripotent embryonic stem cells derived from porcine and bovine species which assertedly are useful for the production of transgenic animals. Further, Wheeler et al, WO 94/26884, published Nov. 24, 1994, disclosed embryonic stem cells which are assertedly useful for the manufacture of chimeric and transgenic ungulates. Thus, based on the foregoing, it is evident that many groups have attempted to produce ES cell lines, e.g., because of their potential application in the production of cloned or transgenic embryos and in nuclear transplantation.

The use of ungulate ICM cells for nuclear transplantation has also been reported. For example, Collas et al., Mol. Reprod. Dev., 38:264-267 (1994) disclose nuclear transplantation of bovine ICMs by microinjection of the lysed donor cells into enucleated mature oocytes. The reference disclosed culturing of embryos in vitro for seven days to produce fifteen blastocysts which, upon transferral into bovine recipients, resulted in four pregnancies and two births. Also, Keefer et al., Biol. Reprod., 50:935-939 (1994), disclose the use of bovine ICM cells as donor nuclei in nuclear transfer procedures, to produce blastocysts which, upon transplantation into bovine recipients, resulted in several live offspring. Further, Sims et al., Proc. Natl. Acad. Sci., USA, 90:6143-6147 (1993), disclosed the production of calves by transfer of nuclei from short-term in vitro cultured bovine ICM cells into enucleated mature oocytes.

Also, the production of live lambs following nuclear transfer of cultured embryonic disc cells has been reported (Campbell et al., Nature, 380:64-68 (1996)). Still further, the use of bovine pluripotent embryonic cells in nuclear transfer and the production of chimeric fetuses has also been reported (Stice et al., Biol. Reprod., 54:100-110 (1996)); Collas et al, Mol. Reprod. Dev., 38:264-267 (1994).

Also, there have been previous attempts to produce cross species NT units (Wolfe et al., Theriogenology, 33:350 (1990). Specifically, bovine embryonic cells were fused with bison oocytes to produce some cross species NT units possibly having an inner cell mass. However, embryonic cells, not adult cells were used, as donor nuclei in the nuclear transfer procedure. The dogma has been that embryonic cells are more easily reprogrammed than adult cells. This dates back to earlier NT studies in the frog (review by DiBerardino, Differentiation, 17:17-30 (1980)). Also, this study involved very phylogenetically similar animals (cattle nuclei and bison oocytes). By contrast, previously when more diverse species were fused during NT (cattle nuclei into hamster oocytes), no inner cell mass structures were obtained. Further, it has never been previously reported that the inner cell mass cells from NT units could be used to form an ES cell-like colony that could be propagated.

Also, Collas et al (Id.), taught the use of granulosa cells (adult somatic cells) to produce bovine nuclear transfer embryos. However, unlike the present invention, these experiments did not involve cross-species nuclear transfer. Also, unlike the present invention ES-like cell colonies were not obtained.

Therefore, notwithstanding what has previously been reported in the literature, there exists a need for improved methods of producing embryonic or stem-like cells. In particular, there exists a need for producing human embryonic or stem-like cells given their significant therapeutic and diagnostic potential.

In this regard, numerous human diseases have been identified which may be treated by cell transplantation. For example, Parkinson's disease is caused by degeneration of dopaminergic neurons in the substantia nigra. Standard treatment for Parkinson's involves administration of L-DOPA, which temporarily ameliorates the loss of dopamine, but causes severe side effects and ultimately does not reverse the progress of the disease. A different approach to treating Parkinson's, which promises to have broad applicability to treatment of many brain diseases and central nervous system injury, involves transplantation of cells or tissues from fetal or neonatal animals into the adult brain. Fetal neurons from a variety of brain regions can be incorporated into the adult brain. Such grafts have been shown to alleviate experimentally induced behavioral deficits, including complex cognitive functions, in laboratory animals. Initial test results from human clinical trials have also been promising. However, supplies of human fetal cells or tissue obtained from miscarriages is very limited. Moreover, obtaining cells or tissues from aborted fetuses is highly controversial.

There is currently no available procedure for producing “fetal-like” cells from the patient. Further, allograft tissue is not readily available and both allograft and xenograft tissue are subject to graft rejection. Moreover, in some cases, it would be beneficial to make genetic modifications in cells or tissues before transplantation. However, many cells or tissues wherein such modification would be desirable do not divide well in culture and most types of genetic transformation require rapidly dividing cells.

There is therefore a clear need in the art for a supply of human embryonic or stem-like undifferentiated cells for use in transplants and cell and gene therapies.

### OBJECTS OF THE INVENTION

It is an object of the invention to provide novel and improved methods for producing embryonic or stem-like cells.

It is a more specific object of the invention to provide a novel method for producing embryonic or stem-like cells which involves transplantation of the nucleus of a mammalian or human cell into an enucleated oocyte of a different species.

It is another specific object of the invention to provide a novel method for producing human embryonic or stem-like cells which involves transplantation of the nucleus of a human cell into an enucleated animal oocyte, preferably an ungulate enucleated oocyte.

It is another object of the invention to provide a novel method for producing human embryonic or stem-like cells which involves transplantation of nuclei of a human cell, e.g., a human adult cell into an enucleated human oocyte.

It is another object of the invention to provide embryonic or stem-like cells produced by transplantation of nuclei of an animal or human cell into an enucleated oocyte of a different species.

It is a more specific object of the invention to provide human embryonic or stem-like cells produced by transplantation of the nucleus of a human cell into an enucleated animal oocyte, preferably an ungulate enucleated oocyte.

It is another object of the invention to use such embryonic or stem-like cells for therapy or diagnosis.

It is a specific object of the invention to use such human embryonic or stem-like cells for treatment or diagnosis of any disease wherein cell, tissue or organ transplantation is therapeutically or diagnostically beneficial.

It is another specific object of the invention to use the embryonic or stem-like cells produced according to the invention for the production of differentiated cells, tissues or organs.

It is a more specific object of the invention to use the human embryonic or stem-like cells produced according to the invention for the production of differentiated human cells, tissues or organs.

It is another specific object of the invention to use the embryonic or stem-like cells produced according to the invention for the production of genetically engineered embryonic or stem-like cells, which cells may be used to produce genetically engineered or transgenic differentiated human cells, tissues or organs, e.g., having use in gene therapies.

It is another specific object of the invention to use the embryonic or stem-like cells produced according to the invention in vitro, e.g. for study of cell differentiation and for assay purposes, e.g. for drug studies.

It is another object of the invention to provide improved methods of transplantation therapy, comprising the usage of isogenic or synegenic cells, tissues or organs produced from the embryonic or stem-like cells produced according to the invention. Such therapies include by way of example treatment of diseases and injuries including Parkinson's, Huntington's, Alzheimer's, ALS, spinal cord injuries, multiple sclerosis, muscular dystrophy, diabetes, liver diseases, heart disease, cartilage replacement, burns, vascular diseases, urinary tract diseases, as well as for the treatment of immune defects, bone marrow transplantation, cancer, among other diseases.

It is another object of the invention to use the transgenic or genetically engineered embryonic or stem-like cells produced according to the invention for gene therapy, in particular for the treatment and/or prevention of the diseases and injuries identified, supra.

It is another object of the invention to use the embryonic or stem-like cells produced according to the invention or transgenic or genetically engineered embryonic or stem-like cells produced according to the invention as nuclear donors for nuclear transplantation.

With the foregoing and other objects, advantages and features of the invention that will become hereinafter apparent, the nature of the invention may be more clearly understood by reference to the following detailed description of the preferred embodiments of the invention and to the appended claims.

### BRIEFS DESCRIPTION OF THE FIGURES

FIG. 1 is a photograph of a nuclear transfer (NT) unit produced by transfer of an adult human cell into an enucleated bovine oocyte.

FIGS. 2 to 5 are photographs of embryonic stem-like cells derived from a NT unit such as is depicted in FIG. 1.

### DETAILED DESCRIPTION OF THE INVENTION

The present invention provides a novel method for producing embryonic or stem-like cells, and more specifically human embryonic or stem-like cells by nuclear transfer or nuclear transplantation. In the subject application, nuclear transfer or nuclear transplantation or NT are used interchangeably.

As discussed supra, the isolation of embryonic or stem-like cells by nuclear transfer or nuclear transplantation has never been reported. Rather, previous reported isolation of ES-like cells has been from fertilized embryos. Also, successful nuclear transfer involving cells or DNA of genetically dissimilar species, or more specifically adult cells or DNA of one species and oocytes of another species has never been reported. Also, to Applicants' knowledge, there has never been reported a method for producing human embryonic or stem-like cells in tissue culture. Rather, the limited human fetal cells and tissues which are currently available must be obtained from spontaneous abortion tissues and from aborted fetuses.

Also, prior to the present invention, no one obtained embryonic or stem-like cells by cross-species nuclear transplantation.

Quite unexpectedly, the present inventors discovered that human embryonic or stem-like cells and cell colonies may be obtained by transplantation of the nucleus of a human cell, e.g., an adult differentiated human cell, into an enucleated animal oocyte, which is used to produce nuclear transfer (NT) units, the cells of which upon culturing give rise to human embryonic or stem-like cells and cell colonies. This result is highly surprising because it is the first demonstration of effective cross-species nuclear transplantation, i.e., the transplantation of cell nuclei from an animal or human cell, e.g., adult cell, into the enucleated egg of a different animal species, to produce nuclear transfer units containing cells which when cultured under appropriate conditions give rise to embryonic or stem-like cells and cell colonies.

Preferably, the NT units used to produce ES-like cells will be cultured to a size of at least 2 to 400 cells, preferably 4 to 128 cells, and most preferably to a size of at least about 50 cells.

In the present invention, embryonic or stem-like cells refer to cells produced according to the present invention. The present invention refers to such cells as stem-like cells rather than stem cells because of the manner in which they are produced, i.e., by cross-species nuclear transfer. While these cells are expected to possess similar differentiation capacity as normal stem cells they may possess some insignificant differences because of the manner they are produced. For example, these stem-like cells may possess the mitochondria of the oocytes used for nuclear transfer.

The present discovery was made based on the observation that nuclear transplantation of the nucleus of an adult human cell, specifically a human epithelial cell obtained from the oral cavity of a human donor, when transferred into an enucleated bovine oocyte, resulted in the formation of nuclear transfer units, the cells of which upon culturing gave rise to human stem-like or embryonic cells and human embryonic or stem-like cell colonies. It is hypothesized by the present inventors that bovine oocytes and human oocytes must undergo maturation processes which are sufficiently similar to permit the bovine oocyte to function as an effective substitute or surrogate for a human oocyte.

Based on the fact that human cell nuclei can be effectively transplanted into bovine oocytes, it is reasonable to expect that human cells may be transplanted into oocytes of other species, e.g., other ungulates as well as other animals. In particular, other ungulate oocytes should be suitable, e.g. pigs, sheep, horses, goats, etc. Also, oocytes from other sources should be suitable, e.g. oocytes derived from other primates, amphibians, rodents, rabbits, etc. Further, using similar methods, it should be possible to transfer human cells or cell nuclei into human oocytes.

Therefore, in its broadest embodiment, the present invention involves the transplantation of an animal or human cell nucleus or animal or human cell into the enucleated oocyte of an animal species different from the donor nuclei, by injection or fusion, to produce an NT unit, containing cells which may be used to obtain embryonic or stem-like cells and/or cell cultures. For example, the invention may involve the transplantation of an ungulate cell nucleus or ungulate cell into an enucleated oocyte of another species, e.g., another ungulate or non-ungulate, by injection or fusion, which cells and/or nuclei are combined to produce NT units and which are cultured under conditions suitable to obtain multicellular NT units, preferably comprising at least about 2 to 400 cells, more preferably 4 to 128 cells, and most preferably at least about 50 cells. The cells of such NT units may be used to produce embryonic or stem-like cells or cell colonies upon culturing.

However, the preferred embodiment of the invention comprises the production of human embryonic or stem-like cells by transplantation of the nucleus of a donor human cell or a human cell into an enucleated animal oocyte, preferably an ungulate oocyte, and most preferably a bovine enucleated oocyte.

In general, the embryonic or stem-like cells will be produced by a nuclear transfer process comprising the following steps:

(i) obtaining desired human or animal cells to be used as a source of donor nuclei;

(ii) obtaining oocytes from a suitable source, e.g. a mammal and most preferably an ungulate, e.g. bovine,

(iii) enucleating said oocytes;

(iv) transferring the human or animal cell or nucleus into the enucleated oocyte of an animal species different than the donor cell or nuclei, e.g., by fusion or injection;

(v) culturing the resultant NT product or NT unit to produce multiple cell structures; and

(vi) culturing cells obtained from said embryos to obtain embryonic or stem-like cells and stem-like cell colonies.

Nuclear transfer techniques or nuclear transplantation techniques are known in the literature and are described in many of the references cited in the Background of the Invention. See, in particular, Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, which are incorporated by reference in their entirety herein. Also, U.S. Pat. Nos. 4,944,384 and 5,057,420 describe procedures for bovine nuclear transplantation.

Human or animal cells may be obtained by well known methods. Human and animal cells useful in the present invention include, by way of example, epithelial, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the human cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

In the example which follows the cells used as donors for nuclear transfer were epithelial cells derived from the oral cavity of a human donor. However, as discussed, the disclosed method is applicable to other human cells or nuclei. Moreover, the cell nuclei may be obtained from both human somatic and cells.

It is also possible to arrest donor cells at mitosis before nuclear transfer, using a suitable technique known in the art. Methods for stopping the cell cycle at various stages have been thoroughly reviewed in U.S. Pat. No. 5,262,409, which is herein incorporated by reference. In particular, while cycloheximide has been reported to have an inhibitory effect on mitosis (Bowen and Wilson (1955) J. Heredity 45: 3-9), it may also be employed for improved activation of mature bovine follicular oocytes when combined with electric pulse treatment (Yang et al. (1992) Biol. Reprod. 42 (Suppl. 1): 117).

Zygote gene activation is associated with hyperacetylation of Histone H4. Trichostatin-A has been shown to inhibit histone deacetylase in a reversible manner (Adenot et al. Differential H4 acetylation of paternal and maternal chromatin precedes DNA replication and differential transcriptional activity in pronuclei of 1-cell mouse embryos. Development (Nov. 1997) 124(22): 4615-4625; Yoshida et al. Trichostatin A and trapoxin: novel chemical probes for the role of histone acetylation in chromatin structure and function. Bioessays (May, 1995) 17(5): 423-430), as have other compounds.

For instance, butyrate is also believed to cause hyper-acetylations of histones by inhibiting histone deacetylase. Generally, butyrate appears to modify gene expression and in almost all cases its addition to cells in culture appears to arrest cell growth. Use of butyrate in this regard is described in U.S. Pat. No. 5,681,718, which is herein incorporated by reference. Thus, donor cells may be exposed to Trichostatin-A or another appropriate deacetylase inhibitor prior to fusion, or such a compound may be added to the culture media prior to genome activation.

Additionally, demethylation of DNA is thought to be a requirement for proper access of transcription factors to DNA regulatory sequences. Global demethylation of DNA from the eight-cell stage to the blastocyst stage in preimplantation embryos has previously been described (Stein et al. Stage-dependent redistributions of acetylated histones in nuclei of the early implantation mouse embryo. Mol. Repro. & Dev. (Aug. 1997) 47(4): 421-429). Jaenisch et al. have reported that 5-azacytidine can be used to reduce the level of DNA methylation in cells, potentially leading to increased access of transcription factors to DNA regulatory sequences. Accordingly, donor cells may be exposed to 5-azacytidine (5-Aza) previous to fusion, or 5-Aza may be added to the culture medium from the 8 cell stage to blastocyst.

The oocytes used for nuclear transfer may be obtained from animals including mammals and amphibians. Suitable mammalian sources for oocytes include sheep, bovines, ovines, pigs, horses, rabbits, guinea pigs, mice, hamsters, rats, primates, etc. In the preferred embodiments, the oocytes will be obtained from ungulates and most preferably bovine.

Methods for isolation of oocytes are well known in the art. Essentially, this will comprise isolating oocytes from the ovaries or reproductive tract of a mammal or amphibian, e.g., a bovine. A readily available source of bovine oocytes is slaughterhouse materials.

For the successful use of techniques such as genetic engineering, nuclear transfer and cloning, oocytes must generally be matured in vitro before these cells may be used as recipient cells for nuclear transfer, and before they can be fertilized by the sperm cell to develop into an embryo. This process generally requires collecting immature (prophase I) oocytes from animal ovaries, e.g., bovine ovaries obtained at a slaughterhouse and maturing the oocytes in a maturation medium prior to fertilization or enucleation until the oocyte attains the metaphase II stage, which in the case of bovine oocytes generally occurs about 18-24 hours post-aspiration. For purposes of the present invention, this period of time is known as the “maturation period.” As used herein for calculation of time periods, “aspiration” refers to aspiration of the immature oocyte from ovarian follicles.

Additionally, metaphase II stage oocytes, which have been matured in vivo have been successfully used in nuclear transfer techniques. Essentially, mature metaphase II oocytes are collected surgically from either non-superovulated or superovulated cows or heifers 35 to 48 hours past the onset of estrus or past the injection of human chorionic gonadotropin (hCG) or similar hormone.

The stage of maturation of the oocyte at enucleation and nuclear transfer has been reported to be significant to the success of NT methods. (See e.g., Prather et al., Differentiation, 48, 1-8, 1991). In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially cattle, the oocyte activation period generally ranges from about 16-52 hours, preferably about 28-42 hours post-aspiration.

For example, immature oocytes may be washed in HEPES buffered hamster embryo culture medium (HECM) as described in Seshagine et al., Biol. Reprod., 40, 544-606, 1989, and then placed into drops of maturation medium consisting of 50 microliters of tissue culture medium (TCM) 199 containing 10% fetal calf serum which contains appropriate gonadotropins such as luteinizing hormone (LH) and follicle stimulating hormone (FSH), and estradiol under a layer of lightweight paraffin or silicon at 39° C.

After a fixed time maturation period, which ranges from about 10 to 40 hours, and preferably about 16-18 hours, the oocytes will be enucleated. Prior to enucleation the oocytes will preferably be removed and placed in HECM containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in HECM, optionally containing 7.5 micrograms per milliliter cytochalasin B, for immediate enucleation, or may be placed in a suitable medium, for example CR1aa, plus 10% estrus cow serum, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in HECM, and then viewing the oocytes under ultraviolet irradiation for less than 10 seconds. The oocytes that have been successfully enucleated can then be placed in a suitable culture medium, e.g., CR1aa plus 10% serum.

In the present invention, the recipient oocytes will preferably be enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation.

A single animal or human cell which is heterologous to the enucleated oocyte will then be transferred into the perivitelline space of the enucleated oocyte used to produce the NT unit. The animal or human cell and the enucleated oocyte will be used to produce NT units according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Essentially, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Graham, Wister Inot. Symp. Monogr., 9, 19, 1969).

Also, in some cases (e.g. with small donor nuclei) it may be preferable to inject the nucleus directly into the oocyte rather than using electroporation fusion. Such techniques are disclosed in Collas and Barnes, Mol. Reprod. Dev., 38:264-267 (1994), and incorporated by reference in its entirety herein.

Preferably, the human or animal cell and oocyte are electrofused in a 500 μm chamber by application of an electrical pulse of 90-120 V for about 15 μsec, about 24 hours after initiation of oocyte maturation. After fusion, the resultant fused NT units are then placed in a suitable medium until activation, e.g., CRIaa medium. Typically activation will be effected shortly thereafter, typically less than 24 hours later, and preferably about 4-9 hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock or cycloheximide treatment may also be used to activate NT embryos after fusion. Suitable oocyte activation methods are the subject of U.S. Pat. No. 5,496,720, to Susko-Parrish et al., which is herein incorporated by reference.

Additionally, activation may be effected by simultaneously or sequentially:

(i) increasing levels of divalent cations in the oocyte, and

(ii) reducing phosphorylation of cellular proteins in the oocyte.

This will generally be effected by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators.

Phosphorylation may be reduced by known methods, e.g., by the addition of kinase inhibitors, e.g., serine-threonine kinase inhibitors such as 6-dimethylaminopurine staurosporine, 2-aminopurine, and sphingosine.

Alternatively, phosphorylation of cellular proteins may be inhibited by introduction of a phosphatase into the oocyte, e.g., phosphatase 2A and phosphatase 2B.

In the preferred embodiment, NT activation will be effected by briefly exposing the fused NT unit to a TL-HEPES medium containing 5 μM ionomycin and 1 mg/ml BSA, followed by washing in TL-HEPES containing 30 mg/ml BSA within about 24 hours after fusion, and preferably about 4 to 9 hours after fusion.

The activated NT units may then be cultured in a suitable in vitro culture medium until the generation of embryonic or stem-like cells and cell colonies. Culture media suitable for culturing and maturation of embryos are well known in the art. Examples of known media, which may be used for bovine embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 MM Ma pyruvate and 50 μg/ml gentamicin sulphate. Any of the above may also involve co-culture with a variety of cell types such as granulosa cells, oviduct cells, BRL cells and uterine cells and STO cells.

In particular, human epithelial cells of the endometrium secrete leukemia inhibitory factor (LIF) during the preimplantation and implantation period. Therefore, the addition of LIF to the culture medium could be of importance in enhancing the in vitro development of the reconstructed embryos. The use of LIF for embronic or stem-like cell cultures has been described in U.S. Pat. No. 5,712,156, which is herein incorporated by reference.

Another maintenance medium is described in U.S. Pat. No. 5,096,822 to Rosenkrans, Jr. et al., which is incorporated herein by reference. This embryo medium, named CR1, contains the nutritional substances necessary to support an embryo.

CR1 contains hemicalcium L-lactate in amounts ranging from 1.0 mM to 10 mM, preferably 1.0 mM to 5.0 mM. Hemicalcium L-lactate is L-lactate with a hemicalcium salt incorporated thereon. Hemicalcium L-lactate is significant in that a single component satisfies two major requirements in the culture medium: (i) the calcium requirement necessary for compaction and cytoskeleton arrangement; and (ii) the lactate requirement necessary for metabolism and electron transport. Hemicalcium L-lactate also serves as valuable mineral and energy source for the medium necessary for viability of the embryos.

Advantageously, CR1 medium does not contain serum, such as fetal calf serum, and does not require the use of a co-culture of animal cells or other biological media, i.e., media comprising animal cells such as oviductal cells. Biological media can sometimes be disadvantageous in that they may contain microorganisms or trace factors which may be harmful to the embryos and which are difficult to detect, characterize and eliminate.

Examples of the main components in CR1 medium include hemicalcium L-lactate, sodium chloride, potassium chloride, sodium bicarbonate and a minor amount of fatty-acid free bovine serum albumin (Sigma A-6003). Additionally, a defined quantity of essential and non-essential amino acids may be added to the medium. CR1 with amino acids is known by the abbreviation “CR1aa.”

CR1 medium preferably contains the following components in the following quantities:

| | | |
|---|---|---|
| sodium chloride | 114.7 | mM |
| potassium chloride | 3.1 | mM |
| sodium bicarbonate | 26.2 | mM |
| hemicalcium L-lactate | 5 | mM |
| fatty-acid free BSA | 3 | mg/ml |

In the preferred embodiment, the activated NT embryos unit will be placed in CR1aa medium containing 1.9 mM DMAP for about 4 hours followed by a wash in HECM and then cultured in CR1aa containing BSA.

For example, the activated NT units may be transferred to CRIaa culture medium containing 2.0 mM DMAP (Sigma) and cultured under ambient conditions, e.g., about 38.5° C., 5% CO₂ for a suitable time, e.g., about 4 to 5 hours.

Afterward, the cultured NT unit or units are preferably washed and then placed in a suitable media, e.g., CRIaa medium containing 10% FCS and 6 mg/ml contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

In the preferred embodiment, the feeder cells will comprise mouse embryonic fibroblasts. Means for preparation of a suitable fibroblast feeder layer is described in the example which follows and is well within the skill of the ordinary artisan.

The NT units are cultured on the feeder layer until the NT units reach a size suitable for obtaining cells which may be used to produce embryonic stem-like cells or cell colonies. Preferably, these NT units will be cultured until at least about 2 to 400 cells, more preferably about 4 to 128 cells, and most preferably at least about 50 cells. The culturing will be effected under suitable conditions, i.e., about 38.5° C. and 5% CO₂, with the culture medium changed in order to optimize growth typically about every 2-5 days, preferably about every 3 days.

In the case of human cell/enucleated bovine oocyte derived NT units, sufficient cells to produce an ES cell colony, typically on the order of about 50 cells, will be obtained about 12 days after initiation of oocyte activation. However, this may vary dependent upon the particular cell used as the nuclear donor, the species of the particular oocyte, and culturing conditions. One skilled in the art can readily ascertain visually when a desired sufficient number of cells has been obtained based on the morphology of the cultured NT units.

After NT units of the desired size are obtained, the cells are mechanically removed from the zone and are then used to produce embryonic or stem-like cells and cell lines. This is preferably effected by taking the clump of cells which comprise the NT unit, which typically will contain at least about 50 cells, washing such cells, and plating the cells onto a feeder layer, e.g., irradiated fibroblast cells. Typically, the cells used to obtain the stem-like cells or cell colonies will be obtained from the inner most portion of the cultured NT unit which is preferably at least 50 cells in size. However, NT units of smaller or greater cell numbers as well as cells from other portions of the NT unit may also be used to obtain ES-like cells and cell colonies.

It may be that a longer exposure of donor cell DNA to the oocyte's cytosol would facilitate the dediffer-entiation process. In this regard, recloning could be accomplished by taking blastomeres from a reconstructed embryo and fusing them with a new enucleated oocyte. Alternatively, the donor cell may be fused with an enucleated oocyte and 4 to 6 hours later, without activation, chromosomes may be removed anf fused with a younger oocyte. Activation would occur thereafter.

The cells are maintained in the feeder layer in a suitable growth medium, e.g., alpha MEM supplemented with 10% FCS and 0.1 mM beta-mercaptoethanol (Sigma) and L-glutamine. The growth medium is changed as often as necessary to optimize growth, e.g., about every 2-3 days.

This culturing process results in the formation of embryonic or stem-like cells or cell lines. In the case of human cell/bovine oocyte derived NT embryos, colonies are observed by about the second day of culturing in the alpha MEM medium. However, this time may vary dependent upon the particular nuclear donor cell, specific oocyte and culturing conditions. One skilled in the art can vary the culturing conditions as desired to optimize growth of the particular embryonic or stem-like cells.

The embryonic or stem-like cells and cell colonies obtained will typically exhibit an appearance similar to embryonic or stem-like cells of the species used as the nuclear cell donor rather than the species of the donor oocyte. For example, in the case of embryonic or stem-like cells obtained by the transfer of a human nuclear donor cell into an enucleated bovine oocyte, the cells exhibit a morphology more similar to mouse embryonic stem cells than bovine ES-like cells.

More specifically, the individual cells of the human ES-line cell colony are not well defined, and the perimeter of the colony is refractive and smooth in appearance. Further, the cell colony has a longer cell doubling time, about twice that of mouse ES cells. Also, unlike bovine and porcine derived ES cells, the colony does not possess an epithelial-like appearance.

The resultant embryonic or stem-like cells and cell lines, preferably human embryonic or stem-like cells and cell lines, have numerous therapeutic and diagnostic applications. Most especially, such embryonic or stem-like cells may be used for cell transplantation therapies. Human embryonic or stem-like cells have application in the treatment of numerous disease conditions.

In this regard, it is known that mouse embryonic stem (ES) cells are capable of differentiating into almost any cell type, e.g., hematopoietic stem cells. Therefore, human embryonic or stem-like cells produced according to the invention should possess similar differentiation capacity. The embryonic or stem-like cells according to the invention will be induced to differentiate to obtain the desired cell types according to known methods. For example, the subject human embryonic or stem-like cells may be induced to differentiate into hematopoietic stem cells, muscle cells, cardiac muscle cells, liver cells, cartilage cells, epithelial cells, urinary tract cells, etc., by culturing such cells in differentiation medium and under conditions which provide for cell differentiation. Medium and methods which result in the differentiation of embryonic stem cells are known in the art as are suitable culturing conditions.

For example, Palacios et al, Proc. Natl. Acad. Sci., USA, 92:7530-7537 (1995) teaches the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferral of cell aggregates to a substrate which provides for cell attachment.

Moreover, Pedersen, J. Reprod. Fertil. Dev., 6:543-552 (1994) is a review article which references numerous articles disclosing methods for in vitro differentiation of embryonic stem cells to produce various differentiated cell types including hematopoietic cells, muscle, cardiac muscle, nerve cells, among others.

Further, Bain et al, Dev. Biol., 168:342-357 (1995) teaches in vitro differentiation of embryonic stem cells to produce neural cells which possess neuronal properties. These references are exemplary of reported methods for obtaining differentiated cells from embryonic or stem-like cells. These references and in particular the disclosures therein relating to methods for differentiating embryonic stem cells are incorporated by reference in their entirety herein.

Thus, using known methods and culture medium, one skilled in the art may culture the subject embryonic or stem-like cells to obtain desired differentiated cell types, e.g., neural cells, muscle cells, hematopoietic cells, etc.

In addition, the use of inducible Bcl-2 or Bcl-xl might be useful for enhancing in vitro development of specific cell lineages. In vivo, Bcl-2 prevents many, but not all, forms of apoptotic cell death that occur during lymphoid and neural development. A thorough discussion of how Bcl-2 expression might be used to inhibit apoptosis of relevent cell lineages following transfection of donor cells is disclosed in U.S. Pat. No. 5,646,008, which is herein incorporated by reference.

The subject embryonic or stem-like cells may be used to obtain any desired differentiated cell type. Therapeutic usages of such differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by fusing adult somatic cells of a cancer or AIDS patient, e.g., epithelial cells or lymphocytes with an enucleated oocyte, e.g., bovine oocyte, obtaining embryonic or stem-like cells as described above, and culturing such cells under conditions which favor differentiation, until hematopoietic stem cells are obtained. Such hematopoietic cells may be used in the treatment of diseases including cancer and AIDS.

Alternatively, adult somatic cells from a patient with a neurological disorder may be fused with an enucleated animal oocyte, e.g., a bovine oocyte, human embryonic or stem-like cells obtained therefrom, and such cells cultured under differentiation conditions to produce neural cell lines. Specific diseases treatable by transplantation of such human neural cells include, by way of example, Parkinson's disease, Alzheimer's disease, ALS and cerebral palsy, among others. In the specific case of Parkinson's disease, it has been demonstrated that transplanted fetal brain neural cells make the proper connections with surrounding cells and produce dopamine. This can result in long-term reversal of Parkinson's disease symptoms.

To allow for specific selection of differentiated cells, donor cells may be transfected with selectable markers expressed via inducible promoters, thereby permitting selection or enrichment for particular cell lineages when differentiation is induced. For example, CD34-neo may be used for selection of hematopoietic cells, Pw1-neo for muscle cells, Mash-1-neo for sympathetic neurons, Ma1-neo for human CNS heurons of the grey matter of the cerebral cortex, etc.

The great advantage of the subject invention is that it provides an essentially limitless supply of isogenic or synegenic human cells suitable for transplantation. Therefore, it will obviate the significant problem associated with current transplantation methods, i.e., rejection of the transplanted tissue which may occur because of host-vs-graft or graft-vs-host rejection. Conventionally, rejection is prevented or reduced by the administration of anti-rejection drugs such as cyclosporine. However, such drugs have significant adverse side-effects, e.g., immunosuppression, carcinogenic properties, as well as being very expensive. The present invention should eliminate, or at least greatly reduce, the need for anti-rejection drugs.

Other diseases and conditions treatable by isogenic cell therapy include, by way of example, spinal cord injuries, multiple sclerosis, muscular dystrophy, diabetes, liver diseases, i.e., hypercholesterolemia, heart diseases, cartilage replacement, burns, foot ulcers, gastrointestinal diseases, vascular diseases, kidney disease, urinary tract disease, and aging related diseases and conditions.

Also, human embryonic or stem-like cells produced according to the invention may be used to produce genetically engineered or transgenic human differentiated cells. Essentially, this will be effected by introducing a desired gene or genes, which may be heterologous, or removing all or part of an endogenous gene or genes of human embryonic or stem-like cells produced according to the invention, and allowing such cells to differentiate into the desired cell type. A preferred method for achieving such modification is by homologous recombination because such technique can be used to insert, delete or modify a gene or genes at a specific cite or cites in the stem-like cell genome.

This methodology can be used to replace defective genes, e.g., defective immune system genes, cystic fibrosis genes, or to introduce genes which result in the expression of therapeutically beneficial proteins such as growth factors, lymphokines, cytokines, enzymes, etc. For example, the gene encoding brain derived growth factor may be introduced into human embryonic or stem-like cells, the cells differentiated into neural cells and the cells transplanted into a Parkinson's patient to retard the loss of neural cells during such disease.

Previously, cell types transfected with BDNF varied from primary cells to immortalized cell lines, either neural or non-neural (myoblast and fibroblast) derived cells. For example, astrocytes have been transfected with BDNF gene using retroviral vectors, and the cells grafted into a rat model of Parkinson's disease (Yoshimoto et al., Brain Research, 691:25-36, (1995)).

This ex vivo therapy reduced Parkinson's-like symptoms in the rats up to 45% 32 days after transfer. Also, the tyrosine hydroxylase gene has been placed into astrocytes with similar results (Lundberg et al., Develop. Neurol., 139:39-53 (1996) and references cited therein).

However, such ex vivo systems have problems. In particular, retroviral vectors currently used are down-regulated in vivo and the transgene is only transiently expressed (review by Mulligan, Science, 260:926-932 (1993)). Also, such studies used primary cells, astrocytes, which have finite life span and replicate slowly. Such properties adversely affect the rate of transfection and impede selection of stably transfected cells. Moreover, it is almost impossible to propagate a large population of gene targeted primary cells to be used in homologous recombination techniques.

By contrast, the difficulties associated with retroviral systems should be eliminated by the use of human embryonic or stem-like cells. It has been demonstrated previously by the subject assignee that cattle and pig embryonic cell lines can be transfected and selected for stable integration of heterologous DNA. Such methods are described in commonly assigned U.S. Ser. No. 08/626,054, filed Apr. 1, 1996, incorporated by reference in its entirety. Therefore, using such methods or other known methods, desired genes may be introduced into the subject human embryonic or stem-like cells, and the cells differentiated into desired cell types, e.g., hematopoietic cells, neural cells, pancreatic cells, cartilage cells, etc.

Genes which may be introduced into the subject embryonic or stem-like cells include, by way of example, epidermal growth factor, basic fibroblast growth factor, glial derived neurotrophic growth factor, insulin-like growth factor (I and II), neurotrophin-3, neurotrophin-4/5, ciliary neurotrophic factor, AFT-1, cytokine genes (interleukins, interferons, colony stimulating factors, tumor necrosis factors (alpha and beta), etc.), genes encoding therapeutic enzymes, etc.

In addition, it is also possible to use one of the negative selection systems now known in the art for eliminating therapeutic cells from a patient if necessary. For example, donor cells transfected with the thymidine kinase (TK) gene will lead to the production of embryonic cells containing the TK gene. Differentiation of these cells will lead to the isolation of therapeutic cells of interest which also express the TK gene. Such cells may be selectively eliminated at any time from a patient upon gancyclovir administration. Such a negative selection system is described in U.S. Pat. No. 5,698,446 for retroviral transfection, and is herein incorporated by reference.

The subject embryonic or stem-like cells, preferably human cells, may be used as an in vitro model of differentiation, in particular for the study of genes which are involved in the regulation of early development.

Also, differentiated cell tissues and organs using the subject embryonic or stem-like cells may be used in drug studies.

Further, the subject embryonic or stem-like cells may be used as nuclear donors for the production of other embryonic or stem-like cells and cell colonies.

In order to more clearly describe the subject invention, the following examples are provided.

### EXAMPLE 1

MATERIALS AND METHODS

Donor Cells for Nuclear Transfer

Epithelial cells were lightly scraped from the inside of the mouth of a consenting adult with a standard glass slide. The cells were washed off the slide into a petri dish containing phosphate buffered saline without Ca or Mg. The cells were pipetted through a small-bore pipette to break up cell clumps into a single cell suspension. The cells were then transferred into a microdrop of TL-HEPES medium containing 10% fetal calf serum (FCS) under oil for nuclear transfer into enucleated cattle oocytes.

Nuclear Transfer Procedures

Basic nuclear transfer procedures have been described previously. Briefly, after slaughterhouse oocytes were matured in vitro the oocytes were stripped of cumulus cells and enucleated with a beveled micropipette at approximately 18 hours post maturation (hpm). Enucleation was confirmed in TL-HEPES medium plus bisbenzimide (Hoechst 33342, 3 μg/ml; Sigma). Individual donor cells were then placed into the perivitelline space of the recipient oocyte. The bovine oocyte cytoplasm and the donor nucleus (NT unit) are fused together using electrofusion techniques. One fusion pulse consisting of 90 V for 15 μsec was applied to the NT unit. This occurred at 24 hours post-initiation of maturation (hpm) of the oocytes. The NT units were placed in CR1aa medium until 28 hpm.

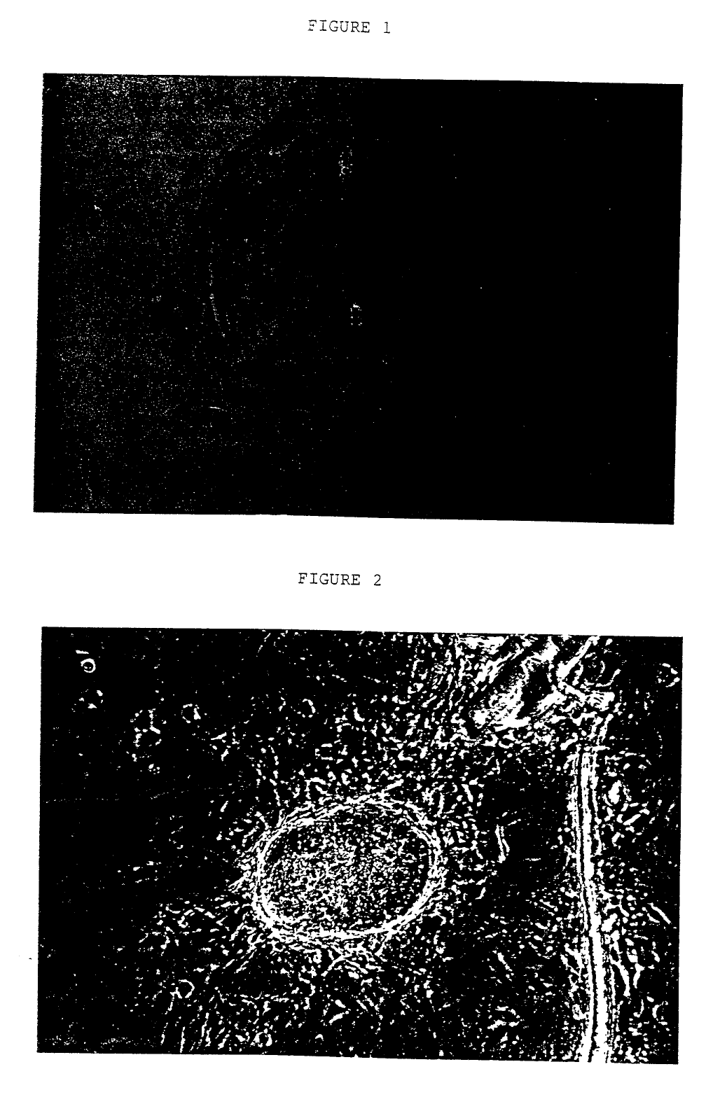

The procedure used to artificially activate oocytes has been described elsewhere. NT unit activation was at 28 hpm. A brief description of the activation procedure is as follows: NT units were exposed for four min to ionomycin (5 μM; CalBiochem, La Jolla, Calif.) in TL-HEPES supplemented with 1 mg/ml BSA and then washed for five min in TL-HEPES supplemented with 30 mg/ml BSA. The NT units were then transferred into a microdrop of CR1aa culture medium containing 0.2 mM DMAP (Sigma) and cultured at 38.5° C. 5% CO₂ for four to five hours. The NT units were washed and then placed in a CR1aa medium plus 10% FCS and 6 mg/ml BSA in four well plates containing a confluent feeder layer of mouse embryonic fibroblasts (described below). The NT units were cultured for three more days at 38.5° C. and 5% CO₂. The culture medium was changed every three days until day 12 after the time of activation. At this time NT units reaching the desired cell number, i.e., about 50 cell number, were mechanically removed from the zona and used to produce embryonic cell lines. A photograph of an NT unit obtained as described above is contained in FIG. 1.

Fibroblast feeder layer

Primary cultures of embryonic fibroblasts were obtained from 14-16 day old murine fetuses. After the head, liver, heart and alimentary tract were aseptically removed, the embryos were minced and incubated for 30 minutes at 37° C. in prewarmed trypsin EDTA solution (0.05% trypsin/0.02% EDTA; GIBCO, Grand Island, N.Y.). Fibroblast cells were plated in tissue culture flasks and cultured in alpha-MEM medium (BioWhittaker, Walkersville, Md.) supplemented with 10% fetal calf serum (FCS) (Hyclone, Logen, Utah), penicillin (100 IU/ml) and streptomycin (50 μl/ml). Three to four days after passage, embryonic fibroblasts, in 35×10 Nunc culture dishes (Baxter Scientific, McGaw Park, Ill.), were irradiated. The irradiated fibroblasts were grown and maintained in a humidified atmosphere with 5% CO₂ in air at 37° C. The culture plates which had a uniform monolayer of cells were then used to culture embryonic cell lines.

Production of embryonic cell line.

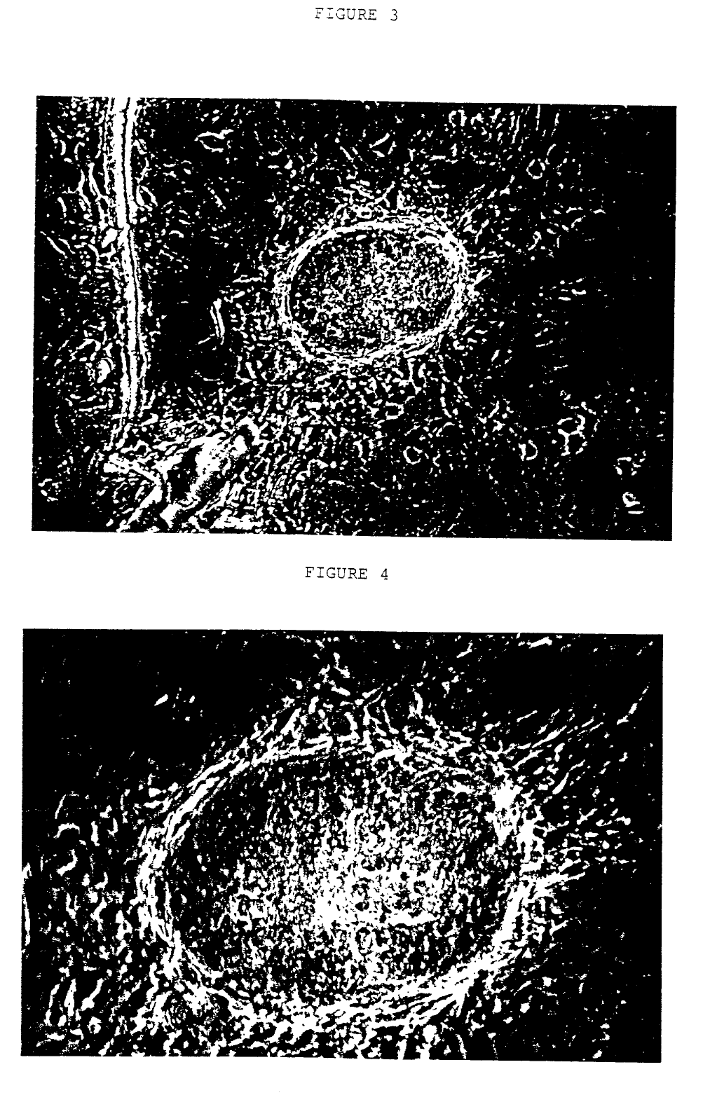

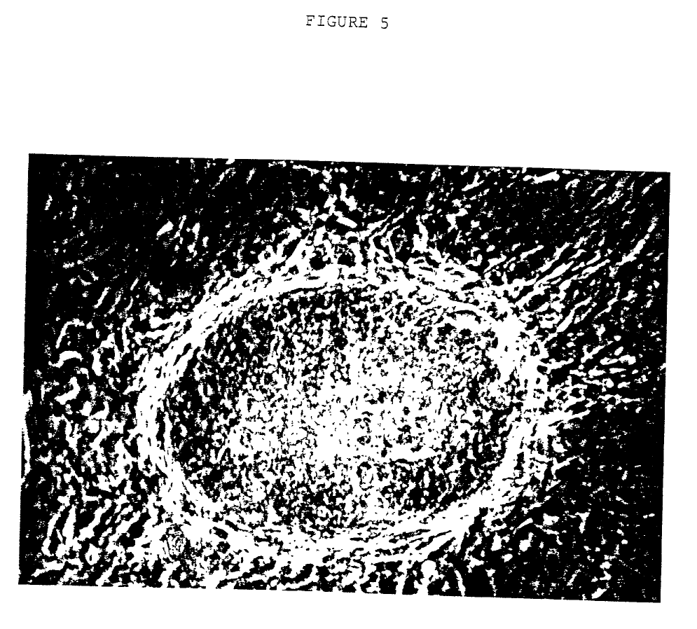

NT unit cells obtained as described above were washed and plated directly onto irradiated feeder fibroblast cells. These cells included those of the inner portion of the NT unit. The cells were maintained in a growth medium consisting of alpha MEM supplemented with 10% FCS and 0.1 mM beta-mercaptoethanol (Sigma). Growth medium was exchanged every two to three days. The initial colony was observed by the second day of culture. The colony was propagated and exhibits a similar morphology to previously disclosed mouse embryonic stem (ES) cells. Individual cells within the colony are not well defined and the perimeter of the colony is refractile and smooth in appearance. The cell colony appears to have a slower cell doubling time than mouse ES cells. Also, unlike bovine and porcine derived ES cells, the colony does not have an epithelial appearance thus far. FIGS. 2 through 5 are photographs of ES-like cell colonies obtained as described, supra.

Production of Differentiated Human Cells

The human embryonic cells obtained are transferred to a differentiation medium and cultured until differentiated human cell types are obtained.

RESULTS Table 1. Human cells as donor nuclei in NT unit production and development.

TABLE 1

| | No. NT | | No. NT units | No. NT units |
|---|---|---|---|---|
| | units | No. NT units 2 | to 4-16 cell | to 16-400 |
| Cell type | made | cell state (%) | stage (%) | cell stage (%) |
| Lymphocytes | 18 | 12 (67%) | 3 (17%) | 0 |
| oral cavity | 34 | 18 (53%) | 3 (9%) | 1 (3%) |
| epithelium | | | | |
| adult fibro- | 46 | 4 (9%) | 12 (4 cell; | — |
| blasts | | | 26%) | |
| | | | 8 (8-16 cells; | |
| | | | 17.4%) | |

The one NT unit that developed a structure having greater than 16 cells was plated down onto a fibroblast feeder layer. This structure was attached to the feeder layer and started to propagate forming a colony with a ES cell-like morphology (See, e.g., FIG. 2). Moreover, although the 4 to 16 cell stage structures were not used to try and produce an ES cell colony, it has been previously shown that this stage is capable of producing ES or ES-like cell lines (mouse, Eistetter et al., Devel. Growth and Differ, 31:275-282 (1989); Bovine, Stice et al., 1996)). Therefore, it is expected that 4-16 cell stage NT units should also give rise to embryonic or stem-like cells and cell colonies.

While the present invention has been described and illustrated herein by reference to various specific materials, procedures, and examples, it is understood that the invention is not restricted to the particular material, combinations of materials, and procedures selected for that purpose. Numerous variations of such details can be implied and will be appreciated by those skilled in the art.

What is claimed is:

1. A method of producing embryonic or stem-like cells comprising the following steps:
(i) inserting a desired human or mammalian cell or cell nucleus into an enucleated animal oocyte, wherein such oocyte is derived from a different animal species than the human or mammalian cell under conditions suitable for the formation of a nuclear transfer (NT) unit;
(ii) activating the resultant nuclear transfer units;
(iii) culturing said activated nuclear transfer units until greater than the 2-cell developmental stage; and
(iv) culturing cells obtained from said cultured NT units to obtain embryonic or stem-like cells.

2. The method of claim 1, wherein the cell inserted into the enucleated animal oocyte is a human cell.

3. The method of claim 2, wherein said human cell is an adult cell.

4. The method of claim 2, wherein said human cell is an epithelial cell, lymphocyte or fibroblast.

5. The method of claim 2, wherein the oocytes are obtained from a mammal.

6. The method of claim 5, wherein the animal oocyte is obtained from an ungulate.

7. The method of claim 6, wherein said ungulate is selected from the group consisting of bovine, ovine, porcine, equine, capine, and buffalo.

8. The method of claim 1, wherein the enucleated oocyte is matured prior to enucleation.

9. The method of claim 1, wherein the fused nuclear transfer units are activated by exposure to ionomycin and DMAP.

10. The method of claim 1, wherein the activated nuclear transfer units are cultured on a feeder layer culture.

11. The method of claim 10, wherein the feeder layer comprises fibroblasts.

12. The method of claim 1, wherein in step (iv) cells from a NT unit having 16 cells or more are cultured on a feeder cell layer.

13. The method of claim 12, wherein said feeder cell layer comprises fibroblasts.

14. The method of claim 13, wherein said fibroblasts comprise mouse embryonic fibroblasts.

15. The method of claim 1, wherein the resultant embryonic or stem-like cells are induced to differentiate.

16. The method of claim 2, wherein the resultant embryonic or stem-like cells are induced to differentiate.

17. The method of claim 1, wherein fusion is effected by electrofusion.

18. Embryonic or stem-like cells obtained according to the method of claim 1.

19. Human embryonic or stem-like cells obtained according to the method of claim 2.

20. Human embryonic or stem-like cells obtained according to the method of claim 3.

21. Human embryonic or stem-like cells obtained according to the method of claim 4.

22. Human embryonic or stem-like cells obtained according to the method of claim 6.

23. Human embryonic or stem-like cells obtained according to the method of claim 7.

24. Differentiated human cells obtained by the method of claim 16.

25. The differentiated human cells of claim 24, which are selected from the group consisting of neural cells, hematopoietic cells, pancreatic cells, muscle cells, cartilage cells, urinary cells, liver cells, spleen cells, reproductive cells, skin cells, intestinal cells, and stomach cells.

26. A method of therapy which comprises administering to a patient in need of cell transplantation therapy isogenic differentiated human cells according to claim 24.

27. The method of claim 26, wherein said cell transplantation therapy is effected to treat a disease or condition selected from the group consisting of Parkinson's disease, Huntington's disease, Alzheimer's disease, ALS, spinal cord defects or injuries, multiple sclerosis, muscular dystrophy, cystic fibrosis, liver disease, diabetes, heart disease, cartilage defects or injuries, bums, foot ulcers, vascular disease, urinary tract disease, AIDS and cancer.

28. The method of claim 26, wherein the differentiated human cells are hematopoietic cells or neural cells.

29. The method of claim 26, wherein the therapy is for treatment of Parkinson's disease and the differentiated cells are neural cells.

30. The method of claim 26, wherein the therapy is for the treatment of cancer and the differentiated cells are hematopoietic cells.

31. The differentiated human cells of claim 24, which contain and express an inserted gene.

32. The method of claim 1, wherein a desired gene is inserted, removed or modified in said embryonic or stem-like cells.

33. The method of claim 32, wherein the desired gene encodes a therapeutic enzyme, a growth factor or a cytokine.

34. The method of claim 32, wherein said embryonic or stem-like cells are human embryonic or stem-like cells.

35. The method of claim 32, wherein the desired gene is removed, modified or deleted by homologous recombination.
